(19) United States
(12) Patent Application Publication
(10) Pub. No.: US 2012/0045798 A1
(43) Pub. Date: Feb. 23, 2012

(54) METHOD AND DEVICE FOR ELECTROWETTING GENETIC ANALYSIS

(75) Inventors: Dorothee Jary, Sassenage (FR); Christine Peponnet, Seyssinet (FR); Valérie Van Wilder, Genoble (FR)

(73) Assignee: Commissariat a l energie atomique et aux energies alternatives, Paris (FR)

(21) Appl. No.: 13/131,021

(22) PCT Filed: Nov. 19, 2009
(86) PCT No.: PCT/EP2009/065507

§ 371 Date: Oct. 21, 2011

(30) Foreign Application Priority Data
Nov. 24, 2008 (FR) 0857944

(51) Int. Cl. C12P 19/34; C12M 1/40

(52) U.S. Cl. 435/91.2; 435/289.1

(57) ABSTRACT

It relates to a method for amplifying at least one nucleotide molecule contained in a sample (E) comprising the following successive steps consisting of (a) subjecting said sample (E) to a 1st amplification step on an area (Z1) of a support; (b) bringing by electrowetting at least one portion of the sample (Ea) obtained after step (a) from the area (Z1) onto at least one area (Z2) of said support, distinct from the area (Z1); (c) subjecting the sample (Ea) to a 2nd amplification step on said area (Z2). It also relates to a device which may be applied within the scope of said method.

### TECHNICAL FIELD

The present invention belongs to the field of molecular biology and more particularly, to the field of amplification of nucleic acids.

The present invention proposes a miniaturized transportable device, easy to make and use and a method applying such a device, both allowing amplification of nucleic acids from samples and notably from samples of small volume.

### STATE OF THE PRIOR ART

The polymerase chain reaction technique or PCR technique is a molecular biologic technique allowing amplification in vitro of nucleic sequences. This technique based on thermal cycles with a denaturation step and a hybridization-elongation step began to be used at the end of the eighties. It is now a standard and indispensable technique which applies to all fields of analysis in which nucleic acids play a direct or indirect role. These fields notably encompass academic research; clinical and diagnostic research and analyses, with tests conducted on blood samples or cell or tissue biopsies; environmental monitoring; civil monitoring; food safety and notably quality control; criminal trace analyses . . . .

The present tools for routine PCR with notably a plate with wells, a heating system and a detection system are relatively bulky and do not allow development of totally portable systems for analysis on site. They do not either allow development of very rapid analysis systems since the constitutive materials such as plastic and the volume of each analysis often of the order of tens of μL, entail incompressible kinetics, during changes in temperature.

Research in the field of PCR aims at developing this technique towards miniaturization both at the level of the sample to be analyzed by PCR and at the level of the actual analysis. Indeed, such miniaturization would allow operations on samples of very small volume, would give the possibility of being less invasive during their sampling, of obtaining a fast method and of having more compact and advantageously portable tools for on-site analysis. Also, there exists strong research activity in the field of PCR microsystems. Such research activity is notably developed by the Fluidigm corporation.

Further, at the present time, it is impossible to conduct many analyses involving PCR from a same sample of reduced volume, unless complicated tools are applied, which are awkward to use, or still under development, such as the pipeting robot Mosquito™ from TTP LabTech (United Kingdom) and Ampligrid™ A480F slides from Advalytix (Germany). Multiple analyses for a same sample of reduced volume may only be contemplated with microsystems but none of them provide a nucleic acid pre-enrichment phase, which may lead to quite poor sensitivity thresholds since the analyzed sample is very small.

International application WO 02/20845 (Thomas Jefferson University) aims at solving this sensitivity problem and proposes a two-step PCR method with which it is possible to prevent the formation of primer-dimers. In this method, the first pre-amplification step has at most 10 denaturation and hybridization-elongation cycles, and applies both primers specific to the nucleic acid to be amplified in a small amount (of less than 0.0625 μM), while the second amplification step uses the same primers in a larger amount and this for 30 to 35 cycles. However, the method of international application WO 02/20845 is only interested in the problem of the sensitivity without tackling the problem of multiple analyses for a same sample of reduced volume.

International application WO 2004/051218 (Applera Corporation) describes a method for conducting a two-step PCR with a first multiplex amplification using a set of primer pairs with which different polynucleotide sequences may be amplified and then a second one with addition of primer pairs specific for a given sequence. The greatest disadvantage of this method lies in the requirement for adding reagents, or even forming aliquots of the product of the multiplex amplification reaction and therefore opening the tubes after this first phase. This has a very strong risk as regards contamination phenomena by amplicons, which is well-known in laboratories working with the PCR technique. Moreover, the case of rare samples of very small volume is not targeted in this application.

International application WO 2008/106719 (Corbett Research) proposes a device and a method for amplifying nucleic acid in two steps. The device has a first compartment for the first amplification step which may be a multiplex amplification and several other compartments for the second amplification step. The liquid samples pass from the first compartment to the other compartments by centrifugation, this first compartment being either directly or indirectly connected to the other compartments. Thus, international application WO 2008/106719 solves the problem of the contamination of the samples by avoiding handling operations between both amplification steps but the described device remains awkward to use and cannot be portable since it requires means allowing it to be centrifuged, to be brought to different temperatures and optionally wax valves for interrupting the fluidic communications between compartments.

Therefore there exists an actual need for a method for amplifying nucleotide molecules using an advantageously miniaturized and transportable device, with which a large number of PCR analyses may be conducted in parallel from a same sample such as a sample of reduced volume.

### DISCUSSION OF THE INVENTION

With the present invention it is possible to find a remedy, at least in part, to the drawbacks and technical problems listed above. Indeed, the latter proposes a method and a device for amplifying nucleotide molecules, based on electrowetting which gives the possibility

of applying the amplification method on site,

of conducting a large number of analyses in parallel from the same sample and notably from a same [symbol] micro-sample [symbol] , i.e. a sample of small or reduced volume, and this without losing any sensitivity.

More particularly, the present invention proposes a method for amplifying at least one nucleotide molecule contained in a sample, designated hereafter as sample (E), comprising the following successive steps:

a) subjecting said sample (E) to a 1^{st} amplification step on a 1^{st} area of a support, designated hereafter as area (Z₁);

b) bringing by electrowetting at least one portion of the sample obtained after step (a), (designated hereafter as sample (Eₐ)), from the area (Z₁) onto at least a 2^{nd} area of said support, distinct from the area (Z₁) and designated hereafter as area (Z₂);

c) subjecting the sample (Eₐ) to a 2^{nd} amplification step on said area (Z₂).

By [symbol] amplification [symbol] , is meant within the scope of the present invention, both a standard amplification by polymerase chain reaction (or PCR) and any PCR alternative known to one skilled in the art such as an asymmetrical PCR, a thermal asymmetrical interlaced PCR, a PCR with a temperature gradient, an end point PCR, a multiplex PCR, a real time PCR, an RT-PCR (Reverse Transcription-Polymerase Chain Reaction [symbol] ) or a quantitative PCR. By [symbol] 1^{st} (or 2^{nd}) amplification step [symbol] , is meant a step consisting in a 1^{st} (or 2^{nd}) amplification, i.e. a 1^{st} (or 2^{nd}) amplification.

By [symbol] RT-PCR [symbol] , is meant reverse transcription followed by polymerase chain amplification. An RT-PCR therefore includes two steps: a reverse transcription step, i.e. for synthesizing a complementary single strand DNA of an RNA sequence, applying a reverse transcriptase followed by a polymerase chain reaction amplification step.

By [symbol] multiplex PCR [symbol] , is meant an amplification method aiming at amplifying more than one amplicon at a time. This technique uses a set of amplification primer pairs, each pair of primers being designed or adapted for amplifying a different nucleotide molecule.

By [symbol] multiplex RT-PCR [symbol] , is meant a multiplex PCR as defined earlier, preceded with reverse transcription as defined earlier.

By [symbol] real time PCR [symbol] , is meant an amplification method which allows detection and/or quantification of the presence of amplicons during the PCR cycles notably by means of a fluorescent marker. The increase in the amplicons or in the signal related to the amount of amplicons formed during the PCR cycles is used for detecting and/or quantifying a given nucleotide sequence in the solution subjected to the PCR.

By [symbol] amplicon [symbol] , is meant a target nucleotide molecule resulting from the PCR amplification of a nucleotide molecule present in the sample (E). The size of the amplicons, within the scope of the present invention, may be comprised between 40 and several thousands of base pairs (bp), notably between 50 and 1,000 bp, in particular, between 60 and 500 bp, and most particularly between 60 and 150 bp.

The expression [symbol] nucleotide molecule [symbol] used herein is equivalent to the following terms and expressions: [symbol] nucleic acid [symbol] , [symbol] polynucleotide [symbol] , [symbol] nucleotide sequence [symbol] , [symbol] polynucleotide sequence [symbol] . By [symbol] nucleotide molecule [symbol] , is meant, within the scope of the present invention, a chromosome; a gene; a regulating polynucleotide; an either single-strand or double-strand, genomic, chromosomal, chloroplastic, plasmid, mitochondrial, recombinant or complementary DNA; a total RNA; a messenger RNA; a ribosomal RNA; a transfer RNA; a portion or a fragment thereof.

The sample (E) applied within the scope of the present invention may be of a very diverse nature. This sample (E) is a sample from an element advantageously selected in the group formed by a liquid solution containing at least one nucleotide molecule as defined earlier; a biological fluid; a plant fluid such as sap, nectar, and root exudate; one or more animal or plant cells; an animal or plant tissue; a food matrix; municipal water, river water, sea water, water from air-cooled towers; an air sample; an earth sample; etc., or one of their mixtures.

The biological fluid is advantageously selected from the group formed by blood such as entire blood or anti-coagulated entire blood, blood serum, blood plasma, lymph, saliva, spittle, tears, sweat, sperm, urine, stools, milk, cerebro-spinal liquid, interstitial liquid, a fluid isolated from bone marrow, mucus, or fluid from the respiratory, intestinal or genito-urinary tract, cell extracts, tissue extracts and organ extracts. Thus, the biological fluid may be any naturally secreted or excreted fluid from a human or animal body or any recovered fluid, from a human or animal body, by any technique known to one skilled in the art such as extraction, sampling or washing. The steps for recovering and isolating these different fluids from the human or animal body are performed prior to the application of the method according to the invention.

The sample (E) applied within the scope of the present invention has a volume comprised between 0.01 and 1,000 μL, notably between 0.02 and 500 μL, in particular, between 0.05 and 100 μL and, more particularly between 0.1 and 20 μL.

The amount of nucleotide molecules contained in the sample (E) is very variable: it may be comprised between 1 and 10¹⁰ nucleotide molecules/sample (E). The nucleotide molecules contained in the sample (E) may be synthetic or natural, stemming from a same organism or from different organisms.

The method according to the present invention applies a device comprising:

a support having at least two distinct areas (Z₁) and (Z₂);

said 1^{st} area (Z₁), designated hereafter by the expression of [symbol] large reservoir [symbol] , being suitable for a first amplification step;

said 2^{nd} area (Z₂) being suitable for a second amplification step;

means allowing a fluid to be brought from said area Z₁ to said area Z₂ by electrowetting. Consequently, the method according to the present invention may comprise a preliminary step for providing such a device or any alternative of this device which will presented hereafter.

Within the scope of the method according to the present invention and prior to step (a) of the latter, the sample (E) is put into contact with the reaction medium required for the amplification of step (a).

The reaction mixture applied during step (a) may be any mixture of commercially available reagents for PCR, such as the kits sold by Roche Applied Science or Applied Biosystems.

Alternatively, the reaction mixture applied during the application of step (a) may have any composition among all the reaction mixtures described in the state of the art for PCR. One skilled in the art will be able to prepare such a reaction mixture depending on the type of PCR applied during step (a) of the method according to the invention.

The reaction mixture comprises one or several elements among a thermostable polymerase such as Taq polymerase and optionally a reverse transcriptase, a salt such as Tris (for trishydroxymethylaminomethane), KCl, NaCl or MgCl₂; deoxyribonucleotide triphosphates such as dATP, dGTP, dTTP, dCTP and optionally dUTP; at least one pair of primers, either specific or degenerate, which may comprise from 15 to 35 base pairs; and optionally at least one oligo-dT or specific primer useful for reverse transcription.

Further, the reaction mixture may contain other additives and notably additives known for improving the efficiency of a PCR such as BSA (Bovine Serum Albumin), betaine, formamide, dimethylsulfoxide, Acetamide or Amide C2, Tween-20, polyethylene glycol (PEG) 6000, proteins such as the [symbol] Single Strand binding protein from E. Coli [symbol] marketed by Sigma Aldrich (increase in the specificity of the reaction), or [symbol] T4 Gene32 Protein from E. Coli B infected with phage T4am134/amBL292/amE219 [symbol] marketed by Roche Applied Science (increase in the production yield of long PCR products, or in the yield of the reaction in the presence of inhibitors such as humic acid), Rnase inhibitors such as [symbol] Ribonuclease Inhibitor [symbol] or diethyl pyrocarbonate which improve the yield of the reverse transcription step during an RT-PCR.

This reaction mixture may contain, when the amplification of step (a) is a multiplex RT-PCR or a multiplex PCR, from 2 to 100 different primer pairs, each pair of primers being specific or degenerate and may comprise from 15 to 35 base pairs.

A reaction mixture example which may be used for the amplification of step (a) of the method according to the present invention comprises between 50 and 100 mM of Tris, between 10 and 100 mM and, advantageously, 50 mM of KCl (or NaCl), between 1 and 5 mM of MgCl₂, between 20 μM and 1 mM of a mixture of dNTPs containing dCTP, dATP, dTTP, dGTP and optionally dUTP, either hot start or not Taq polymerase at 0.1 U/μL, primers comprising between 15 and 35 base pairs and which may be specific or degenerate. Each primer is advantageously present, in this reaction mixture, at a concentration comprised between 1 and 200 nM and, in particular between 10 and 100 nM. Further, it was seen that the obtained results are better when between 0.1 to 1 mg/ml of BSA and of excess Taq Polymerase with respect to the recommendations of the suppliers is added to the reaction mixture. As an example, in the case of TaqGold ABI, a concentration of 0.5 U/μL is used instead of the concentration of 0.1 U/μL recommended by ABI.

The sample (E) which is electrically conducting is brought onto said area (Z₁) by electrowetting and this, in the form of one or several drops. The sample (E) may be directly brought onto the area (Z₁) or onto an area (Z₁₀), distinct from (Z₁) and then onto the area (Z₁).

The contacting between the sample (E) and the reaction mixture prior to step (a) of the method according to the invention may be accomplished in different ways:

it may take place prior to the positioning of the sample (E) on the area (Z₁) of the support, in this case, it is the sample (E) associated with the reaction mixture which is positioned on said area (Z₁) by electrowetting;

it may take place on the area (Z₁) of the support, after or simultaneously with the positioning of the sample (E) on said area (Z₁).

This latter application may have several alternatives. In a first alternative, the reaction mixture is brought by electrowetting advantageously as a drop, as far as the area (Z₁) where it mixes with the sample (E) by coalescence. In this alternative, the reaction mixture may be present:

either on the support and notably on an area (Z₃) of the support, said area (Z₃) being distinct from the areas (Z₁) and (Z₂) defined earlier,

or in a reaction mixture reservoir located on the outside of the device applied within the scope of the present invention, and may be brought onto the support via an orifice passing through a substrate positioned facing said support, said orifice may be connected to the reaction mixture reservoir either directly or indirectly through any entry device such as a capillary or a flex.

In a second alternative, the reaction mixture or at least some of its constituents have been dried or freeze-dried beforehand on area (Z₁) of said support, during its making or subsequently to the latter.

Additives known in the state of the art may be applied for the step for drying or freeze-drying the reaction mixture or some of its constituents such as the primers or probes on the area (Z₁) of the support. These additives are notably selected from sugars or sugar-alcohols such as mannitol, saccharose, trehalose, glucose, etc.; polymers such as dextran, PEG, polyvinylpyrrolidone (PVP) or polyvinyl acetate (PVA) . . . ; or further EDTA. The amount of reagents deposited for drying or freeze-drying may be increased in order to avoid a loss of reagents during the amplification step (a) due to the loss of reagents on the surfaces because of the drying or freeze-drying. It was seen that without increasing the amount of dried constituents of the reaction mixture, the amplification efficiency during step (a) is reduced as compared with the one obtained under standard conditions, i.e. without any dried reagents. Advantageously, the amount of the constituents of the reaction mixture to be dried should be multiplied by a factor of about 2 relatively to the amount used in the absence of any drying or freeze-drying step.

During step (a) of the method according to the present invention, all conditions of temperature and of duration for the different cycles (denaturation, hybridization and elongation), known to one skilled in the art and suitable for the type of PCR used during this step, may be used.

Advantageously, the amplification applied during step (a) of the method according to the invention is selected from the group formed by a multiplex PCR and a multiplex RT-PCR. This step is a step for pre-amplification of the nucleotide molecules contained in the sample (E).

During step (a) of the method, multiplex PCR is carried out with a first denaturation step at 95° C., lasting for 1 second to 10 minutes depending on the size of the nucleotide molecules present in the sample (E) and on the nature of the enzyme used, followed by 5 to 15, advantageously 10 thermal cycles with a denaturation step at 95° C. for 1 to 30 seconds and a hybridization-elongation step at a lower temperature, notably comprised between 50 and 70° C., advantageously 60° C. for 5 to 300 seconds and, in particular, for 5 to 30 seconds.

During the step (a) of the method and within the scope of a multiplex RT-PCR, the reverse transcription step may last for 5 to 90 minutes at 37° C. and the multiplex PCR cycles following this 1^{st} step may be from 5 to 20 in number, advantageously 10 in number. The thermal cycles during the PCR may:

comprise two temperatures with a hybridization-elongation plateau comprised between 50 and 70° C., preferentially 60° C., maintained for 5 to 300 seconds and a denaturation plateau at 95° C. maintained for 1 to 30 seconds;

comprise three temperatures with a hybridization plateau comprised between 50 and 65° C., advantageously 60° C., maintained for 5 to 300 seconds, an elongation plateau between 65° C. and 75° C., advantageously 72° C., maintained for 5 to 300 seconds and a denaturation plateau at 95° C. maintained for 1 to 30 seconds;

or be [symbol] touch down [symbol] cycles consisting of gradually reducing the hybridization temperature by 1° C. per cycle for example.

The temperatures applied at each cycle during the amplification of step (a) of the method according to the invention are attained, maintained and controlled by using means giving the possibility of heating the applied support and notably the area (Z₁) of the latter, and optionally means for controlling the temperature of the applied support and notably of the area(s) (Z₂) of the latter.

The concentration for each pair of primers during the multiplex PCR cycles during step (a) of the method is comprised between 10 and 200 nM, advantageously of the order of 50 nM, and may be different for the different nucleotide molecules to be amplified. It may notably be adjusted if this allows correction of the amplification biases due to competition phenomena well-known for multiplex amplification. The duration of the cycles may also be adapted in order to correct all the competition biases during multiplex PCR. The number of sequences amplified in parallel during this first step may be comprised between 2 and several hundreds, advantageously be of the order of several tens.

The step (b) of the method according to the present invention more particularly consists of forming at least one drop from the sample (Eₐ) obtained after step (a) of the method by electrowetting and of moving said drop from said area (Z₁) to said area (Z₂) by electrowetting.

Advantageously, the step (b) consists of forming by electrowetting, x drop(s) from the sample (Eₐ) obtained after step (a) of the method and of moving, by electrowetting, said drop(s) from said area (Z₁) to y area(s) (Z₂) with x≧y and x and y representing an integer comprised between 1 and 500, advantageously between 1 and 50, notably between 2 and 30 and, in particular, between 2 and 20. The areas (Z₂) of the support are also designated herein by the expression of [symbol] small reservoirs [symbol] . In a particular alternative, x is equal to y.

The drop(s) formed during step (b) of the method has(have) an extremely reduced volume, of the order of one nanoliter. This volume is notably comprised between 0.1 and 100 nL, in particular between 1 and 80 nL and, more particularly, a volume of 10, 30, 50 or 65 nL. In the case when several drops are formed from the sample (Eₐ), the latter may have identical (drops of calibrated volume) or different volume.

It is clear for one skilled in the art that the sample (Eₐ) corresponds to the sample (E) completed with the reaction mixture used for the amplification of step (a) and of the amplicons produced during this first amplification step.

The amplification during (c) of the method according to the present invention is advantageously a simplex PCR, insofar that one mainly has a PCR applying at least one pair of primers specific to a given nucleotide molecule and optionally at least one marked probe specific to said nucleotide molecule.

By [symbol] marked probe [symbol] , is meant, within the scope of the present invention, a fluorescent probe capable of binding either onto the double-strand DNA (SYBR technology) or onto a specific DNA sequence (Taqman and Beacon technology) and which is not fluorescent once it is bound to the DNA. Specific marked probes may be of any kind among probes known and described in the state of the art. As examples of specific marked probes which may be used within the scope of the present invention, mention may be made of TaqMan probes, molecular beacons, Scorpion probes and LNA probes.

Advantageously, step (c) applies from 1 to 4 pairs of primers specific to a given nucleotide molecule (or to a given organism and optionally from 1 to 4 marked probes specific to said nucleotide molecule (or said organism), provided with 1 to 4 different markers.

Within the scope of the present invention, after positioning of at least one drop of sample (Eₐ) on at least one area (Z₂) of the support and prior to step (c) of said method, said drop is put into contact with the specific primer pair(s) and optionally with the specific marked probe(s) required for the amplification of step (c).

The contacting between the sample (Eₐ) and the specific primer pair(s) and optionally the specific marked probe(s) prior to step (c) of the method according to the invention may take place on the area (Z₂) of the support, after or simultaneously with the positioning of the sample (Eₐ) on said area (Z₂).

In a first alternative, an electrically conducting solution (S) containing the specific primer pair(s) and optionally the specific marked probe(s) is brought by electrowetting, advantageously as a drop, as far as the area (Z₂) where it mixes with the sample (Eₐ). In this alternative, said solution (S) may be present:

either on the support and notably on an area (Z₄) of the support, said area (Z₄) being distinct from the areas (Z₁), (Z₂) and (Z₃) defined earlier,

or in a reservoir of solution (S) located outside the device applied within the scope of the present invention, and may be brought onto the support via an orifice crossing the substrate positioned facing said support, said orifice may be connected to the reservoir of solution (S) either directly or indirectly and this, through any entry device such as a capillary or a flex.

In a second alternative, the specific primer pair(s) and optionally the specific marked probe(s) have been dried or freeze-dried beforehand on the area (Z₂) of said support, during its manufacturing or subsequently to the latter.

The particularities described earlier for the step for drying or freeze-drying the reaction mixture or certain of its constituents on the area (Z₁) of the support such as the addition of additives and the amount of reagents are applied mutatis mutandis in the step for drying or freeze-drying the specific primer pair(s) and optionally the specific marked probe(s) on the area (Z₂) of said support. The concentration of specific dried primers and probes after mixing with the sample (Eₐ) drop will be comprised between 100 and 2000 nM, advantageously of the order of 600 nM.

Whether the specific primer pair(s) and optionally the specific marked probe(s) appear in liquid form or in a dried or freeze-dried form, their mixture with the sample (Eₐ) on the area (Z₂) is performed by a out-and-back movement on electrodes adjacent to said area (Z₂).

During step (c) of the method of the present invention, all conditions of temperature and of duration for the different cycles (denaturation, hybridization and elongation), known to one skilled in the art and suitable for PCR amplification with specific primers and optionally specific probes, may be used.

Advantageously, the amplification applied during step (c) of the method according to the invention comprises from 20 to 40 cycles, notably 30, each cycle including a denaturation step and an hybridization-elongation step. Further, the amplification applied during step (c) of the method gives the possibility of obtaining amplicons of reduced size comprising from 50 to 200 bp, and, in particular, from 60 to 150 bp.

The thermal cycles during the amplification applied during step (c) of the method according to the invention may be comprised between 50 and 70° C. for hybridization-elongation, advantageously of the order of 60° C., and be of the order of 95° C. for denaturation. They may last between 5 and 180 seconds, advantageously between 5 and 30 seconds, for hybridization-elongation and between 1 and 30 seconds, advantageously between 1 and 5 seconds for denaturation.

The temperatures applied at each cycle during the amplification of step (c) of the method according to the invention are attained, maintained and controlled by using means giving the possibility of heating the applied support and notably the area(s) (Z₂) of the latter, and optionally means giving the possibility of controlling the temperature of the applied support and notably the area(s) (Z₂) of the latter.

During step (c) of the method according to the invention and when the latter applies at least one specific marked probe, the fluorescence of the drops may be measured at each thermal cycle for real-time analysis or else at the end of step (c) for end point analysis. Fluorescent detection of the amplified DNA uses, during step (c) of the method according to the invention, a fluorophore such as green Sybr of a standard fluorophore for marking PCR probes such as Alexa Fluor488, Alexa Fluor546, Carboxy-Rhodamine 6G, Cy3, Cy5, 6-FAM, Fluorescein, HEX, JOE, TAMRA, TET, Texas Red.

During step (c) of the method according to the invention and when the latter does not apply any specific marked probe, no detection of the amplicons is carried out during this step. The amplicons may require subsequent analysis by another technique such as hybridization on a DNA chip or electrophoresis. In this case, it may be necessary to associate the device applied during the method according to the invention and more particularly the area(s) (Z₂) with another analysis device. Thus, in this alternative, the product obtained after step (c) may be recovered on each area (Z₂) manually or by electrowetting.

In the latter case, at least one drop is formed by electrowetting from the product obtained after step (c) and the latter is, by electrowetting, brought towards a structure such as an orifice crossing the substrate positioned facing the support, an orifice crossing said support or any hybrid device which may be adapted by one skilled in the art allowing manipulation of discrete drops and/or transformation of the sample in the form of drops into a fluidic vein sample.

Depending on the nature of the sample (E) applied within the scope of the present invention and as defined earlier, the method according to the present invention may comprise a preliminary step for purifying the sample (E), the latter involving the preparation of said sample and optionally the isolation of the nucleotide molecules contained in the latter.

This preliminary step may be performed in different ways and may apply detergents leading to lysates, enzymes such as a lysozyme or proteinase K, an ultrasound treatment or mechanical stirring in the presence of beads.

In certain cases, it may be necessary to purify the extracted nucleotide molecules in order to clear them from possible contaminants such as nucleases or PCR-inhibiting molecules. This purification of nucleotide molecules may be achieved by extraction with phenol-chloroform, by chromatography, by ion exchange, by electrophoresis, by centrifugation at equilibrium and/or by capture by hybridization on a solid support. Commercially available purification and extraction cases or kits may be used during the preliminary step for purifying the sample (E).

Thus, the device applied within the scope of the present invention may optionally be interfaced with a module for automatically preparing samples with which a pure solution of nucleotide molecule(s) may be obtained with a volume adapted to said device, i.e. a volume comprised between 0.1 to 20 μL. This sample preparation module may be directly present on the device. In this case, a technique for purifying nucleotide molecules will preferentially be used, based on the use of magnetic particles such as magnetic beads on a laboratory-on-a-chip operating by electrowetting, as described in the literature (Fouillet et al. [symbol] Digital microfluidic design and optimization of classic and new fluidic functions for lab on a chip systems [symbol] 3^{rd} Microfluidics French Conference μFlu2006-23; Yizhong Wang et al., [symbol] Efficient in-droplet separation of magnetic particles for digital microfluidics [symbol] J. Micromech. Microeng. 17 (2007) 2148-2156). This purification technique uses a magnet placed in proximity to the device applied within the scope of the present invention, during steps for capturing magnetic beads. This magnet is moved away during the steps for re-dispersing the beads. Re-dispersion is accomplished by stirring the fluid with displacements of the drops by activating the electrodes.

In a particular embodiment, the method according to the present invention comprises a preliminary step for purifying the sample (E) comprising the steps consisting of:

i) bringing the sample (E) into contact with a lysis solution;

ii) bringing the lyzed sample (E) obtained after step (i) into contact with at least one magnetic bead capable of adsorbing at least one nucleotide molecule;

iii) washing said magnetic bead;

iv) bringing said washed bead obtained after step iii) into contact with an elution solution and optionally heating the thereby obtained solution;

v) isolating the nucleotide molecule(s) obtained after step (iv).

During step (ii), the magnetic beads are advantageously in a saline solution in which the concentration of salts and the pH are optimum for capturing nucleotide molecules on the beads. The magnetic beads used are advantageously obtained commercially and may be generic for capturing nucleotide molecules (Chemicell silanol beads,) or specific molecules (Dynabeads Streptavidin beads), or any other type of beads which may be functionalized with an oligonucleotide.

The compositions of the lysis, washing and elution solutions are well-known to the state of the art and one skilled in the art may prepare them without any inventive effort.

Alternatively, the lysis, washing or elution solutions may be obtained from commercial kits.

Commercial kits for purifying genomic or plasmid DNA, messenger or total or ribosomal RNA may be used (kits developed by Dynal, Polysciences, Novagen, Bilatec, . . . ) by selecting the kit adapted to the type of tested sample (E) and to the targeted application. Achieving purification of nucleic acids on the basis of commercial on-chip kits may however require adaptations: (1) possibly modification of the recommended volumes and (2) possibly addition of surfactant(s) into certain solutions in order to allow the latter to be introduced onto the support applied within the scope of the present invention. As examples of surfactants which may be used, mention may be made of Tween20 or Triton X-100, more particularly, used at final concentrations from 0.05% to 2%.

During the preliminary step for purifying the sample (E), the latter may be positioned on the support, advantageously by electrowetting,

α) prior to step (i) (raw sample (E)),

β) subsequently to the latter and prior to step (ii) (lyzed sample (E)),

γ) subsequently to step (ii) and prior to step (iii) (lyzed sample (E) mixed with magnetic bead(s)).

The sample (E) may be deposited either on the area (Z₁) as defined earlier, or on an area (Z₅) distinct from the areas (Z₁), (Z₂), (Z₃) and (Z₄) as defined earlier. The sample may be brought onto the support via an orifice crossing the substrate positioned facing said support, said orifice may be connected to the sample (E) reservoir either directly or indirectly through any entry device such as a capillary or a flex.

In the alternative (a), the sample (E) deposited on the area (Z₁) or on an area (Z₅) is mixed with the electrically conducting lysis solution, brought on said area by electrowetting and this in the form of one or several drops. Said lysis solution may be present on the support on an area (Z₆) distinct from the areas (Z₁) to (Z₅) as defined earlier or may be brought onto the support via an orifice crossing the substrate positioned facing said support, said orifice may be connected to the lysis solution reservoir either directly or indirectly through any entry device such as a capillary or a flex.

Subsequently or in the alternative (β), the lyzed sample (E), present on the area (Z₁) or (Z₅), is mixed with the electrically conducting solution of magnetic beads, brought on said area by electrowetting and this in the form of one or several drops. Said solution of magnetic beads may be present on the support on an area (Z₇) distinct from the areas (Z₁) to (Z₆) as defined earlier or may brought onto the support via an orifice crossing the substrate positioned facing said support, said orifice may be connected to the magnetic bead solution reservoir either directly or indirectly through any entry device such as a capillary or a flex.

Subsequently or following the alternative (γ), magnetic beads are captured with a magnet in order to form a pellet of magnetic beads and the supernatant is discharged from the device by electrowetting and this, in the form of one or several drops. The supernatant is advantageously directed towards a [symbol] garbage bin [symbol] present on the device. The latter advantageously appears as an orifice crossing the substrate positioned facing the support, an orifice crossing said support or an enclave in the lid. By [symbol] enclave [symbol] is meant that the lid is recessed in its thickness so as to make a larger space between the lid and the support.

Prior to step (iii) for washing the magnetic beads, the electrically conducting washing solution may be brought as far as the pellet of magnetic beads, by electrowetting and this in the form of one or several drops. Said washing solution may be present on the support on an area (Z₈) distinct from the areas (Z₁) to (Z₇) as defined earlier or may be brought via an orifice crossing the substrate positioned facing said support, said orifice may be connected to the washing solution reservoir either directly or indirectly through any entry device such as a capillary or a flex. During step (iii), the magnetic beads may be re-dispersed into the washing solution by a out-and-back movement of the latter, induced by electrowetting.

The magnetic beads may again be recaptured in a compact pellet by bringing the magnet closer to the area (Z₁) or (Z₅). The supernatant is again discharged via the garbage bin as defined earlier. A new washing solution, either identical or different from the previous one, may be put into contact with the pellet of magnetic beads according to the procedure alternatives defined earlier and this for repeating the washing step (iii) at least once. The washing step (iii) may be repeated as many times as required with the same washing solution or with one or several other washing solutions.

Step (iv) consists of bringing into contact an elution solution of nucleotide molecules on the pellet of magnetic beads obtained following the last washing step. The introduction of said electrically conducting elution solution advantageously uses electrowetting. Said washing solution may be present on the support on an area (Z₉) distinct from the areas (Z₁) to (Z₈) as defined earlier and may brought via an orifice crossing the substrate positioned facing said support, said orifice may be connected to the elution solution reservoir either directly or indirectly through any entry device such as a capillary or a flex. During step (iv), the magnetic beads may be re-dispersed in the elution solution by a out-and-back movement of the latter, induced by electrowetting. During step (iv), the elution solution in contact with the dispersed magnetic beads may be heated in order to more efficiently [symbol] detach [symbol] the nucleotide molecules retained on said beads. This heating may involve the use of means with which the applied support may be heated and notably the area (Z₁) or (Z₅) of the latter, and optionally of means with which the temperature of the applied support may be controlled and notably that of the area (Z₁) or (Z₅) of the latter.

Step (v) consists of capturing the magnetic beads cleared of nucleotide molecules in a compact pellet by means of a magnet. The thereby obtained supernatant is the pure solution containing the nucleotide molecules which forms the purified sample (E) applied in step (a) of the method according to the invention. When the purification steps have taken place on the area (Z₅) of the support, prior to applying step (a) of the method according to the invention, the purified sample (E) should be brought onto the area (Z₁) by electrowetting and this in the form of one or several drops.

The present invention also relates to a device which may be applied in a method as defined earlier, said device comprising a support having at least two distinct areas (Z₁) and (Z₂),

said 1^{st} area (Z₁) being adapted for a 1^{st} amplification step;

said 2^{nd} area (Z₂) being adapted for a 2^{nd} amplification step;

means with which a fluid may be brought from said area (Z₁) to said area (Z₂) by electrowetting.

Advantageously, said area (Z₂) of the support has at least one pair of specific primers and optionally at least one specific marked probe as defined earlier, either freeze-dried or dried.

Further, the support may have at least one additional area, distinct from the areas (Z₁) and (Z₂) and selected from the group formed by:

an area (Z₃) on which is positioned a reaction mixture as defined earlier, useful for the first amplification step;

an area (Z₄) on which is positioned a solution S as defined earlier, containing at least one pair of specific primers and optionally at least one specific marked probe as defined earlier, useful for the 2^{nd} amplification step;

an area (Z₅) adapted for purifying a sample such as the sample (E) defined earlier;

an area (Z₆) on which is positioned a lysis solution as defined earlier;

an area (Z₇) on which is positioned a solution containing magnetic beads, as defined earlier;

an area (Z₈) on which is positioned a washing solution as defined earlier, and

an area (Z₉) on which is positioned an elution solution as defined earlier.

The support also designated herein by the term of [symbol] chip [symbol] comprises, in a sectional view, a substrate, having an array of electrodes, an insulating layer (i.e. dielectric layer) covering substrate and electrodes and a hydrophobic layer covering said dielectric layer. The different areas (Z₁) to (Z₉) as defined earlier are therefore located at the surface of said hydrophobic layer.

By [symbol] areas [symbol] is therefore meant mainly an arrangement of electrodes defined at the surface of the chip, each of these electrodes having a particular function and optionally a shape adapted to the function. For example, it is possible to have reservoir electrodes, transfer electrodes for transferring the drops, electrodes dedicated to mixing the reagents, etc . . . .

The support may be made according to the method described in the state of the art (Fouillet Y et al. [symbol] Digital microfluidic design and optimization of classic and new fluidic functions for lab on a chip systems [symbol] Microfluid. Nanofluid. (2008) 4:159-165).

The support has not only means with which a fluid may be brought from the area (Z₁) to the area (Z₂) but also:

means with which a fluid may be brought from the area (Z₄) to the area (Z₂) and

means with which a fluid from an area selected in the group formed by the areas (Z₃), (Z₆), (Z₇), (Z₈) and (Z₉) may be brought to an area selected from between the area (Z₁) and the area (Z₅).

With these means it is possible to manipulate and to displace drops of fluid and apply series of adjacent electrodes in a plane, which may positioned linearly but also in two dimensions and this, for defining a displacement plane of a fluid in the form of drops. In the electrowetting technique, the displacement of the drops also involves a counter-electrode maintaining an electric contact with the drop during displacement and a voltage generator for applying a potential difference between the electrodes and the counter-electrode.

The electrodes may be made by depositing a metal layer such as a layer of Au, Al, ITO, Pt, Cr or Cu by photolithography. The dielectric layer may be in Si₃N₄, in parylene or in SiO₂.

The device of the invention may have an [symbol] open [symbol] figuration. In this case, it only consists of the support as defined earlier and the counter-electrode advantageously appears as a catenary wire.

However, in order to promote portability of the device according to the present invention, the latter advantageously has a [symbol] closed [symbol] configuration. In the latter, the support of the device as defined earlier is associated with a substrate positioned facing said support. This substrate also designated herein by the term of [symbol] lid [symbol] includes conductive means in order to form a counter-electrode. It is advantageously made in a material adapted for fluorescence measurements and has an electric layer and an hydrophobic layer. The hydrophobic layers of the support and of the lid will preferentially be deposited according to the method described in the article of Thery et al., [symbol] SiOC as a hydrophobic layer for electrowetting on dielectric applications [symbol] 11^{th} International Conference on Miniaturized Systems for Chemistry and Life Sciences (MicroTas—07-11.10.2007—Paris France).

The drops positioned on the support displaced on the latter are confined between the support and the substrate positioned facing said support, the space between the support and the substrate being comprised between 10 and 1,000 nm, advantageously between 50 and 100 nm. Taking into account the small volume displaced via the drops, an oil aiming at confining the drops and preventing any evaporation risks may be added. Said oil may for example be a mineral oil such as oil M3516 from Sigma-Aldrich or any other oil which may be adapted by one skilled in the art. In addition to its function related to evaporation, it is important to select oil promoting the displacement of the drops by electrowetting. The oil also has a passive protective role against contamination of the samples.

The substrate has, as already explained, at least one suction orifice or hole which may advantageously have a convergent shape towards the space between the support and the substrate and allowing a liquid to be deposited on the support or its suction from the latter. Indeed, the suction of a liquid on a given area of the support is possible when the electrodes connected to the orifice crossing the lid are activated. Also, the deposit of a liquid on a given area of the support is possible via said orifice when the latter is connected to an injection device.

The device according to the present invention may comprise means allowing the support or at least a given area of this support and more particularly the areas (Z₁), (Z₂) and (Z₅) as defined earlier, to be brought to a given temperature notably during step (a), during step (c) and/or during the step for purifying the sample (E). These means with which the support may be brought to a given temperature may either be a heating element of the Peltier type in contact with the space of the support opposite the face bearing the areas (Z₁), (Z₂) and the possible other areas (Z₃) to (Z₉), or one or more heating resistors integrated to the device.

These integrated heating resistors may be made from a thin metal layer such as a thin layer in platinum, in aluminium, in doped silicon, etc . . . , by defining patterns with standard micro-manufacturing techniques (photolithography and etching for example) or for example by screen printing of a conducting material such as platinum. The design of these heating resistors (thickness, section, length) will be carried out so as to generate a given heating power for a set voltage, while obtaining a well-defined electric resistance value. Advantageously, these heating resistors may allow localization of the heating under the areas of interest, such as the areas (Z₁), (Z₂) and (Z₅), etc . . . , within the scope of the method applied in the invention.

Moreover, the device according to the present invention may also comprise one or more temperature probes with which it is possible to control the temperature of the samples and of the reagents as close as possible to the areas (Z₁), (Z₂) and (Z₅) as defined earlier. The method for manufacturing such temperature probes is identical with the method for manufacturing heating resistors as discussed earlier. For example, it is possible to subordinate the probes to a Peltier block so as to accurately control the temperature of the support.

By definition, the technologies for manufacturing devices using electrowetting involve the steps for defining patterns in metal layers. The making of heating resistors and of temperature probes therefore does not add any complexity in the overall method for manufacturing the device according to the invention.

The device according to the present invention is distinguished from the devices of the prior art, by the fact that:

it is portable since there is miniaturization of the volumes and of the size of the analysis consumable;

it allows very rapid analyses since the temperatures during the PCR amplification steps are rapidly attained, considering the miniaturization of the sample volume to be treated and of the material used for the device, which is a very good heat conductor;

it does not have to be opened between the pre-amplification phase and the amplification phase with specific detection, for adding reagents, or even between the purification of the sample be treated and the amplification phase with specific detection; which avoids all the recognized and very serious risks of contamination by amplicons.

Further, the device may have a large number of areas, such as the areas (Z₅) to (Z₉) defined earlier, and the organization of the areas (Z₁) to (Z₉) defined earlier at the surface of the support and their arrangements relatively to each other are more free and are not imposed unlike the case of the device described in international application WO 2008/106719, in which the arrangement is imposed by the centrifugal force, which moves the liquids from one compartment to another.

It should be noted that the device, once the sample (E) is introduced onto the latter, may no longer be in contact with the environment having a contaminating potential. This is notably the case of a closed device onto which the reagent(s) required for the different PCR amplification steps and for the optional step of purification of the sample to be analyzed has(have) been loaded on board in liquid, dried or freeze-dried form.

Finally, the device according to the invention provides the possibility of amplifying and analyzing at least two different samples, or even 3, 4, or more different samples. FIGS. 1 and 2 propose optimum architectures for amplifying nucleotide molecules for two samples (E1) and (E2) according to the invention (FIG. 1) with optionally a preliminarily step for purifying the nucleotide molecules contained in these samples (FIG. 2).

FIG. 1 proposes a device as seen from above in a [symbol] closed [symbol] configuration, for which only the support is materialized, said device having:

two areas (Z₁₀₋₁) and (Z₁₀₋₂) onto which the samples (E₁) and (E₂) are deposited respectively;

two areas (Z₃₋₁) and (Z₃₋₂) on which are positioned the liquid reagents for the 1^{st} amplification step;

a plurality of reservoirs forming the large reservoirs (Z₁₋₁) and (Z₁₋₂), on which the 1^{st} step is applied for amplifying the samples (E₁) and (E₂) respectively;

2×13 areas (Z₂), on which the 2^{nd} amplification step is applied. On each area (Z₂), specific either identical or different reagents (primers and optionally marked probes) are dried.

With the device having an optimized architecture of FIG. 1, it is possible to perform for two samples (E₁) and (E₂) simultaneously:

1) mixing between the sample and the liquid reagents for the 1^{st} amplification step and sending of the mixture into a large reservoir (Z₁₋₁) or (Z₁₋₂) for the 1^{st} PCR step;

2) the 1^{st} PCR step in two large reservoirs (Z₁₋₁) and (Z₁₋₂) notably in parallel;

3) distributing and sending 65 nL drops at the end of the 1^{st} PCR step;

4) the 2^{nd} PCR step with specific detection in 13 small reservoirs (Z₂).

FIG. 2 proposes a device seen from above, in a [symbol] closed [symbol] configuration, said device having:

two areas (Z₁₀₋₁) and (Z₁₀₋₂) onto which the samples (E₁) and (E₂) are deposited respectively;

two areas (Z₁₋₁) and (Z₁₋₂), on which the purification and the first amplification step of the samples (E₁) and (E₂) respectively are applied;

two areas (Z₃₋₁) and (Z₃₋₂) on which are positioned the liquid reagents for the 1^{st} amplification step;

6 areas (Z_{A}) on which are positioned the liquid reagents for the 1^{st} amplification step and/or the reagents for purifying the samples (E₁) and (E₂), these areas (Z_{A}) may advantageously be selected from the areas (Z₃), (Z₆), (Z₇), (Z₈) and (Z₉) as defined earlier;

an orifice (T₁) on the substrate positioned facing the support, through which the different supernatants obtained during the purification step are discharged;

2×11 areas (Z₂), on which the 2^{nd} amplification step is applied. In each area (Z₂), specific either identical or different reagents (primers and optionally marked probes) are dried.

With the device having an optimized architecture of FIG. 2, it is possible to achieve for two samples (E₁) and (E₂) in parallel, simultaneously:

1) purification of the nucleotide molecules from two samples (E₁) and (E₂);

2) a 1^{st} step for amplifying both purified samples (E₁) and(E₂);

3) a 2^{nd} amplification step with specific probes in 11 small reservoirs for the two purified samples (E₁) and (E₂).

The present invention also relates to an amplification kit comprising:

a device as defined earlier, and

at least one element selected from the group formed by a thermostable polymerase, a reverse transcriptase, deoxyribonucleotide triphosphates, a pair of primers, either specific or degenerate, an oligo-dT or specific primer useful for reverse transcription and a marked probe. Each of these elements is as defined earlier.

The method, the device and the amplification kit according to the present invention allow and are useful in field analyses such as environmental monitoring, civil monitoring, food safety, medical analyses, which may range from taking blood with a syringe to capillary sampling, analyses of cancer markers on microbiopsies, criminal analyses on traces, food processing quality controls, veterinary analyses with notably the search for viral, bacterial, parasitic or mycological pathogens, on capillary samplings or microbiopsies.

Other features and advantages of the present invention will further become apparent to one skilled in the art upon reading the examples below given as an illustration and not as a limitation, with reference to the appended figures.

### SHORT DESCRIPTION OF THE DRAWINGS

FIG. 1 proposes a device with an optimized architecture in order to conduct the analysis of a sample by gene amplification in two steps with pre-amplification of the sample in a closed microcomponent operating by electrowetting.

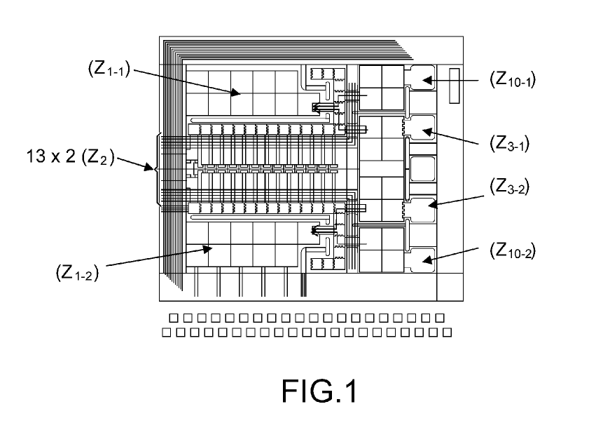

FIG. 2 proposes a device with an optimized architecture for performing purification of nucleic acids of two samples in parallel, followed by analysis by gene amplification in two steps with pre-amplification of the sample in a closed microcomponent operating by electrowetting.

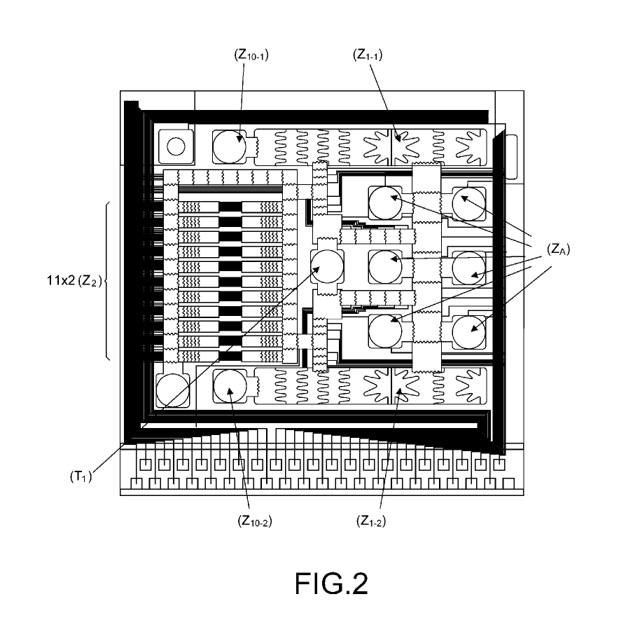

FIG. 3 proposes an illustration of the carrying out of the two-step amplification procedure on a microdevice operating by electrowetting.

FIG. 4 shows the increase in fluorescence obtained in 3 small reservoirs during the second amplification step of the procedure with which the presence of the pathogens E. Coli, Bacillus Subtilis and Adenovirus 2 may be detected in the initial sample.

FIG. 5 shows the supply of reagents, the position of the magnet and of the electrodes connecting the reagent supply area, the purification area and the garbage bin for the purification of nucleic acids on a EWOD chip from a blood sample.

FIG. 6 shows the PCR analysis of purified DNA on an EWOD chip operating by electrowetting from a blood sample.

### DETAILED DISCUSSION OF PARTICULAR EMBODIMENTS

I. Example of a Particular Application of the Method According to the Invention.

1. Sample and Reagents.

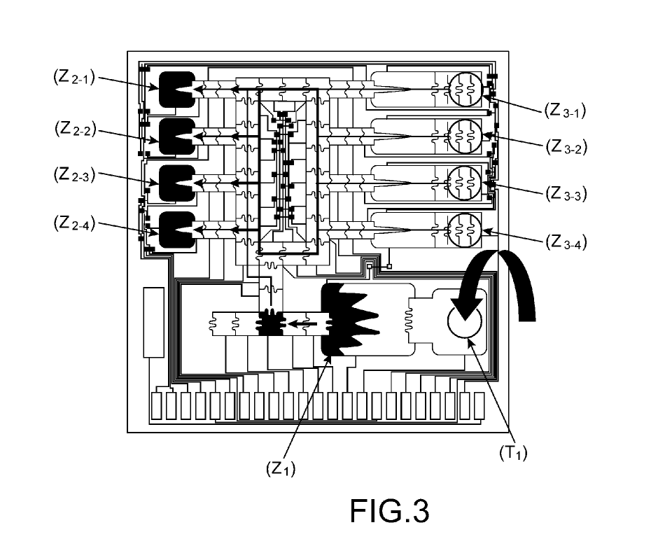

A sample stemming from a preparation of nucleic acids containing 10⁶ copies of the Gram-bacterium Escherichia Coli, 10³ copies of the adenovirus type II and 10³ copies of the bacterium Bacillus Subtilis, is mixed with PCR reagents (BSA 0.8 mg/mL, AmpliTaq Gold enzyme buffer without MgCl₂ 1×, MgCl₂ 3 mM, nucleotides (dATP, dTTP, dCTP, dGTP) 200 μM, betaine 450 mM, AmpliTaq Gold (0.5 U/μL) and primers specific to the studied pathogens at different concentrations (Escherichia Coli 20 nM/Adenovirus 20 nM/Bacillus subtilis 80 nM). In the 4 small storage reservoirs Z₃₋₁, Z₃₋₂, Z₃₋₃ and Z₃₋₄ (FIG. 3) of the microcomponent, the primers and probes specific to each investigated pathogens were loaded on board beforehand (deposits of droplets of solutions onto the spotting robot and then dried). The deposited amounts correspond to a concentration of 600 nM after re-suspension.

2. Carrying Out the Steps.

The sample is first of all mixed in a tube with the PCR reagents for the first amplification step and heated for 10 minutes at 95° C. in a tube (the DNA denaturation and Taq Gold activation step).

This solution is then introduced onto the chip through a hole of the lid (T₁) and by activation of the electrodes. The first step of the cascade PCR is carried out in the large reservoir (Z₁) (FIG. 3): 10 thermal cycles with a denaturation step at 95° C. (5 seconds) and a hybridization-elongation step at 60° C. for 20 seconds.

At the end of this first step, the sample is distributed in 65 nL drops and each drop is sent into the small storage reservoirs Z₂₋₄, Z₂₋₂, Z₂₋₃ and Z₂₋₄. The mixing of the sample drop and of the specific reagents loaded on board beforehand is performed by a out-and-back movement of the drops on electrodes adjacent to the small reservoir (10 round trips).

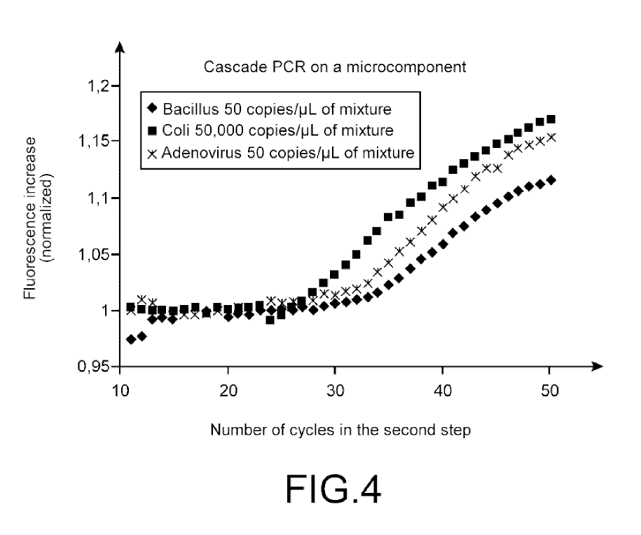

The second cascade PCR step is carried out in the small reservoirs. It consists in 40 thermal cycles with a denaturation step at 95° C. (5 seconds) and a hybridization-elongation step at 60° C. for 20 seconds. During this second PCR step, the fluorescence of the drops is measured at each thermal cycle at the end of the hybridization-elongation step. The fluorescent signal obtained during this second amplification step is given in FIG. 4.

II. Example of Another Application of the Method According to the Invention.

In order to prove that it is possible to couple a preparation of samples directly on the microdevice according to the invention, and a two-step amplification, a DNA purification example on the microdevice is given.

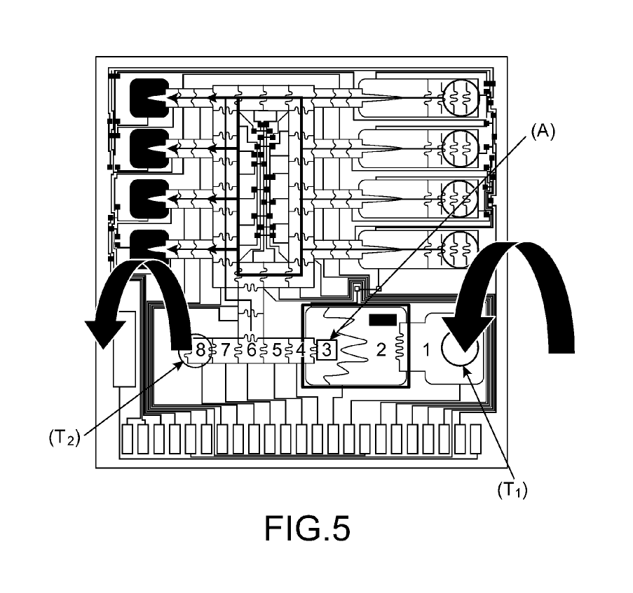

Commercial reagents (Bilatest Genomic DNA kit) were used. A mixture of blood, lysis buffer, binding buffer and magnetic beads was introduced on a chip (total volume of the mixture: 5 μL containing 0.26 μL of blood) through the hole (T₁) (FIG. 5). The electrodes Nos. 1 to 8 are activated in order to displace the solution from the inlet hole (T₁) to the garbage bin formed by the hole (T₂). When the liquid is displaced from the hole (T₁) to the hole (T₂), the beads are collected into a compact pellet when they pass in proximity to the magnet (A) placed on the lid above the electrode 3.

The first washing solution (washing buffer A of the Bilatest Genomic DNA kit) is then deposited in the hole (T₁) with a micro-pipette. It is introduced onto the chip by activating the electrodes 1, 2 and 3 and then by deactivating the electrode 1. The beads are redispersed in the washing solution by moving the magnet (A) away from the lid and by successively activating the electrodes 2 and 3 for 2 minutes. The magnet (A) is then again brought closer to the lid of the chip in order to reform a compact pellet of beads. The supernatant is then brought into the garbage bin formed by the hole (T₂) by activating the electrodes 3, 4, 5, 6, 7 and 8 and then by successively deactivating the electrodes 3, 4, 5, 6, 7 and 8. The whole of these steps for washing the magnetic beads with the washing solution A is repeated three times (FIG. 5).

The second washing solution (washing buffer B of the Bilatest Genomic DNA kit) is then deposited in the hole (T₁) with a micro-pipette. It is introduced onto the chip by activating the electrodes 1, 2 and 3 and then by deactivating the electrode 1. The beads are not redispersed in this washing solution and the supernatant is brought into the garbage bin formed by the hole (T₂) by activating the electrodes 3, 4, 5, 6, and 8 and then by successively deactivating the electrodes 3, 4, 5, 6, 7 and 8.

The DNA elution solution (Bilatest Genomic DNA kit) is then deposited in the hole (T₁) with a micro-pipette. It is introduced onto the chip by activating electrodes 1, 2 and 3 and then by deactivating the electrode 1. The beads are redispersed into the elution solution by moving the magnet (A) away from the lid, by successively activating the electrodes 2 and 3 for 10 minutes and by heating the chip to 55° C. The magnet (A) is then again brought closer to the lid of the chip in order to reform a compact pellet of beads. The supernatant is the solution of purified DNA.

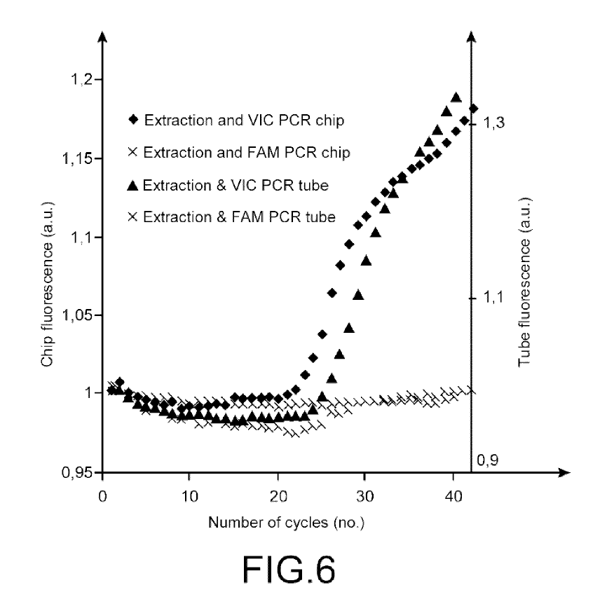

It was checked that the thereby obtained solution on a chip actually contains the DNA at the expected concentration by carrying out real-time genotyping PCR (detection of the deletion ΔF508 of the CFTR gene). We compared the aspect of the fluorescence curves for a purified sample on a chip and a purified sample in a tube by following the procedure provided by Bilatec with the kit (FIG. 6). The obtained curves are absolutely similar in both cases with Ct (for [symbol] Threshold cycle [symbol] , i.e. the cycle from which fluorescence increases significantly and is disconnected from the background noise and by means of which it is possible to trace back the amount of initially present DNA molecules) showing that the concentration of DNA obtained on the chip and in the tube is quite similar (6.8 ng of DNA obtained per μL of purified blood in both cases).

1) A method for amplifying at least one nucleotide molecule contained in a sample, designated hereafter as sample (E), comprising the following successive steps consisting of:
a) subjecting said sample (E) to a 1^{st} amplification step on a 1^{st} area of a support, designated hereafter as area (Z₁);
b) bringing by electrowetting at least one portion of the sample obtained after step (a), designated hereafter as the sample (Eₐ), from the area (Z₁) onto at least one 2^{nd} area of said support, distinct from the area (Z₁) and designated hereafter as (Z₂);
c) subjecting the sample (Eₐ) to a 2^{nd} amplification step on said area (Z₂).

2) The method according to claim 1, characterized in that said sample (E) is a sample of an element selected from the group formed by a liquid solution containing at least one nucleotide molecule; a biological fluid; a plant fluid; one or more animal or plant cells; an animal or plant tissue; a food matrix; municipal water, river water, seawater, water from air-cooled towers; an air sample; an earth sample or one of their mixtures.

3) The method according to any of claim 1 or 2, characterized in that, prior to said step (a), said sample (E) is put into contact with the reaction medium required for the amplification of said step (a).

4) The method according to any of claims 1 to 3, characterized in that said sample (E) is brought onto said area (Z₁) by electrowetting.

5) The method according to any of the preceding claims, characterized in that said amplification during said step (a) is selected from the group formed by multiplex PCR and multiplex RT-PCR.

6) The method according to any of the preceding claims, characterized in that said step (b) consists of forming by electrowetting x drop(s) from said sample (Eₐ) and of displacing, by electrowetting, said drop(s) of said area (Z₁) towards y area(s) (Z₂), with x≧y and x and y representing an integer between 1 and 50.

7) The method according to any of the preceding claims, characterized in that said amplification during said step (c) applies at least one pair of specific primers of a given nucleotide molecule and optionally at least one specific marked probe of said nucleotide molecule.

8) The method according to any of the preceding claims, characterized in that said method comprises a preliminary step for purifying the sample (E).

9) The method according to claim 8, characterized in that said preliminary purification step comprises the steps:
i) bringing the sample (E) into contact with a lysis solution;
ii) bringing the lyzed sample (E) obtained after step (i) into contact with at least one magnetic bead capable of adsorbing at least one nucleotide molecule;
iii) washing said magnetic bead;
iv) bringing said washed bead obtained after step (iii) into contact with an elution solution and optionally heating the thereby obtained solution;
v) isolating the nucleotide molecule(s) obtained after step (iv).

10) A device which may be applied in a method as defined in any of the preceding claims, said device comprising a support having at least two distinct areas (Z₁) and (Z₂),
said 1^{st} area (Z₁) being adapted for a first amplification step;
said 2^{nd} area (Z₂) being adapted for a second amplification step and having at least one pair of specific primers and optionally at least one specific marked probe, either freeze-dried or dried; and
means with which a fluid may be brought from said area (Z₁) to said area (Z₂) by electrowetting.

11) The device according to claim 10, characterized in that the support has at least one additional area, distinct from the areas (Z₁) and (Z₂) and selected from the group formed by:
an area (Z₃) on which is positioned a reaction mixture, useful for the 1^{st} amplification step;
an area (Z₄) on which is positioned a solution S, containing at least one pair of specific primers and optionally at least one specific marked probe, useful for the 2^{nd} amplification step;
an area (Z₅) adapted for purifying a sample;
an area (Z₆) on which is positioned a lysis solution;
an area (Z₇) on which is positioned a solution containing magnetic beads;
an area (Z₈) on which is positioned a washing solution; and
an area (Z₉) on which is positioned an elution solution.

12) The device according to claim 10 or 11, characterized in that said support is associated with a substrate positioned facing said support.

13) The device according to any of claims 10 to 12, characterized in that said device comprises means with which the support or at least one given area of this support may be brought to a given temperature.

14) The device according to any of claims 10 to 13, characterized in that said device comprises at least one temperature probe.

15) An amplification kit comprising:
a device as defined in any of claims 10 to 14, and
at least one element selected from the group formed by a thermostable polymerase, a reverse transcriptase, deoxyribonucleotide triphosphates, a pair of primers, either specific or degenerated, an oligo-dT or specific primer useful for reverse transcription and a marked probe.
